(19) United States
(12) Patent Application Publication
(10) Pub. No.: US 2012/0101330 A1
(43) Pub. Date: Apr. 26, 2012

(54) Implantable system, a device for inserting the implantable system and a method thereof

(75) Inventors: Gershon Goldenberg, Carcur (IL); Eran Hirszowicz, Ramat Gan (IL); Amos Neeman, Moshav Bazra (IL)

(73) Assignee: REALVIEW MEDICAL LIMITED, Misgav (IL)

(21) Appl. No.: 13/263,081

(22) PCT Filed: Apr. 6, 2010
(86) PCT No.: PCT/IL10/00274

§ 371 Date: Dec. 19, 2011

Related U.S. Application Data
(60) Provisional application No. 61/166,850, filed on Apr. 6, 2009.

(51) Int. Cl. A61B 1/04; A61B 1/00

(52) U.S. Cl. 600/109; 600/116

(57) ABSTRACT

A method and an implantable system for insertion into a cavity of a person, comprising: an imaging element capable of acquiring an image of at least a portion of the cavity through an optical element; and a porous inflatable enclosure arranged to enclose the imaging element and to enclose liquid that flows through the porous inflatable enclosure and contacts the optical element, wherein the liquid, when in contact with the optical element, reduces an aggregation of non-transparent material on the optical element while the implantable system is located in the cavity of the person.

### RELATED APPLICATIONS

This application claims the benefit of U.S. provisional patent Ser. No. 61/166,850 filing date Apr. 6, 2010 which is incorporated herein in its entirety.

### FIELD OF THE INVENTION

The invention relates to implantable systems and more specifically to a tiny in-vivo image recording implant.

### BACKGROUND OF THE INVENTION

There is a growing need for in-vivo imaging of cell, tissue or organ at on-demand basis. In view of the progress of tiny image recording cameras, numerous publications suggested implants which capture images or spatial information of body lumens and even tissue regions.

There are two major issues which limit the implantable system retention time in the body: (i) encapsulation or buildup of scar tissue on a viewing aperture of the implantable system retention; and (ii) biofilm growth on the external surface of the implantable system retention.

The growth of biofilm is even more severe since in addition to blurring the viewing aperture, biofilm is the main source for implant originated bacterial contamination. Biofilm growth on implants, and in particular permanent implants, poses a major challenge to the medical community due to its hosted bacteria colonies.

It is particularly important to prevent biofilm growth in imaging implants which are expected to be retained for extended periods within the patient body. Once developed, the biofilm may grow to a level that prevents clear vision. Furthermore, in certain in-vivo environments, such as the urine, the biofilm may serve as a primer layer for developing sediment encrustation layer which completely blocks the implant vision capability.

The functionality of implantable systems may be beneficial as an add-on feature for a device which at least partially dwells within the urinary tract. Such add-on may be used to early detection of various diseases and conditions within the urinary tract.

An implantable system may be beneficial as a tool for a TCC (Transitional Cell Carcinoma) recurrent monitoring following resection. A urinary bladder cancer remains one of the most prevalent of all cancers.

TCC, the most typical urinary bladder cancer, recurs in nearly 70% of patients. After recurrence, urinary bladder tumors may also invade deeper in the urinary bladder wall. An early detection of TCC recurrences substantially increases the 5-year survival rate.

TCC patients typically undergo eight cystoscopy procedures in the two-year period following initial diagnosis. As an invasive, uncomfortable, and costly procedure, cystoscopy requires local or general anesthesia, and provides only limited visibility.

Several non-invasive methods have been suggested for early detection of urinary bladder cancer. However, their detection capability is limited due to high false negative results. As described above, failure to early detect urinary bladder cancer and especially TCC may shorten the patient life expectancy.

Therefore it is highly desired to obtain on-demand imaging capability within the urinary bladder for a period of two years following the resection. One way to provide image coverage of the urinary bladder interior is by using a floating implantable system and guiding it, using magnetic elements and varying magnetic fields.

As stated above, there is a need for an imaging device that can be implanted in a human organ for a prolong period of time without producing contamination and without visibility blocking.

### SUMMARY OF THE INVENTION

An implantable system for insertion into a cavity of a person, the system includes an imaging element capable of acquiring an image of at least a portion of the cavity through an optical element; and a porous inflatable enclosure arranged to enclose the imaging element and to enclose liquid that flows through the porous inflatable enclosure and contacts the optical element, wherein the liquid, when in contact with the optical element, reduces an aggregation of non-transparent material on the optical element while the implantable system is located in the cavity of the person.

An implantable system for insertion into a cavity of a person, the system may include an imaging element; and a porous inflatable enclosure arranged to enclose the imaging element and to enclose liquid that flows through the porous inflatable enclosure, wherein the flow of liquid reduces an attachment of biofilm on the implantable system, when the implantable system is located in the cavity of the person.

A method for imaging a cavity of a person, the method includes: imaging at least a portion of the cavity of the person by an imaging element that belongs to an implantable system that is inserted in the cavity of the person, wherein the imaging comprises acquiring at least one image through an optical element; and flowing fluid through a porous inflatable enclose arranged to enclose the imaging element and to enclose liquid that flows through the porous inflatable enclosure and contacts the optical element, wherein the liquid, when in contact with the optical element, reduces an aggregation of non-transparent material on the optical element while the implantable system is located in the cavity of the person.

A method for imaging a cavity of a person, the method includes: imaging at least a portion of the cavity of the person by an imaging element that belongs to an implantable system that is inserted in the cavity of the person, wherein the imaging element has a field of view; and flowing liquid through a porous inflatable enclosure that encloses the imaging element, wherein the flow of liquid reduces an attachment of a non-transparent biofilm on the implantable system, when the implantable system is located in the cavity of the person.

### BRIEF DESCRIPTION OF THE INVENTION

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

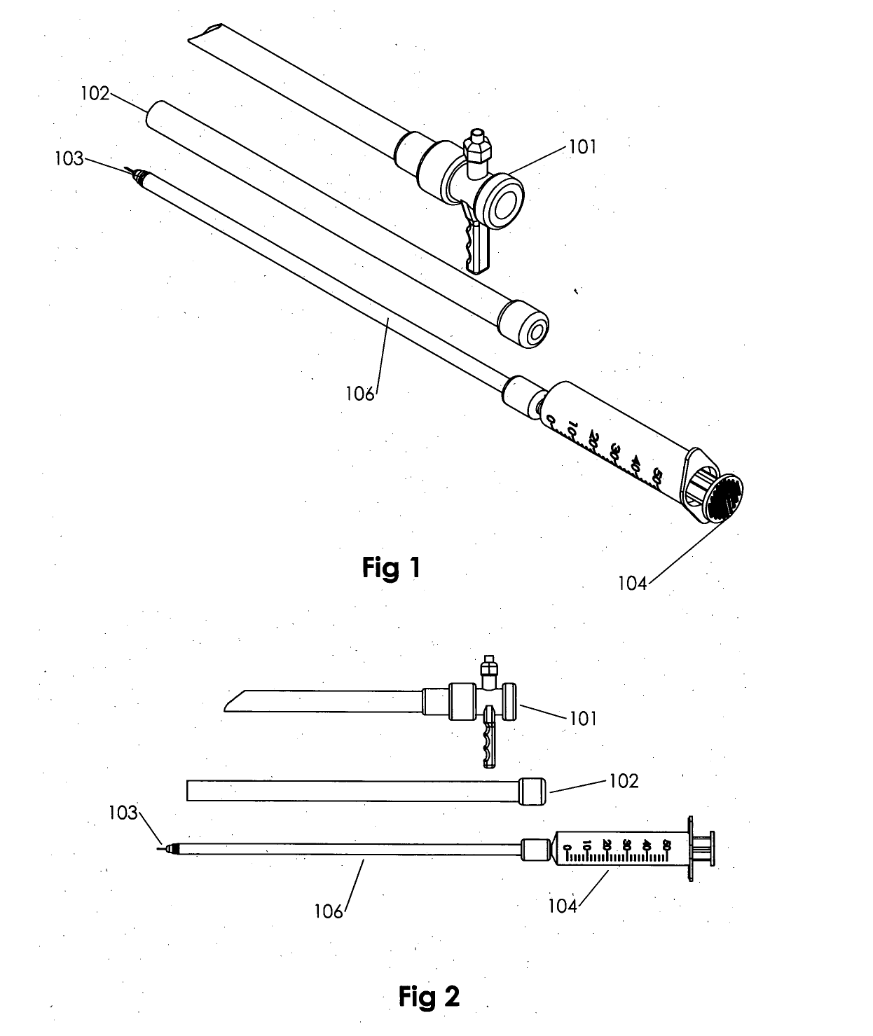

FIGS. 1 and 2 illustrates portions of a device for inserting an implantable system into a cavity of a person according to an embodiment of the invention;

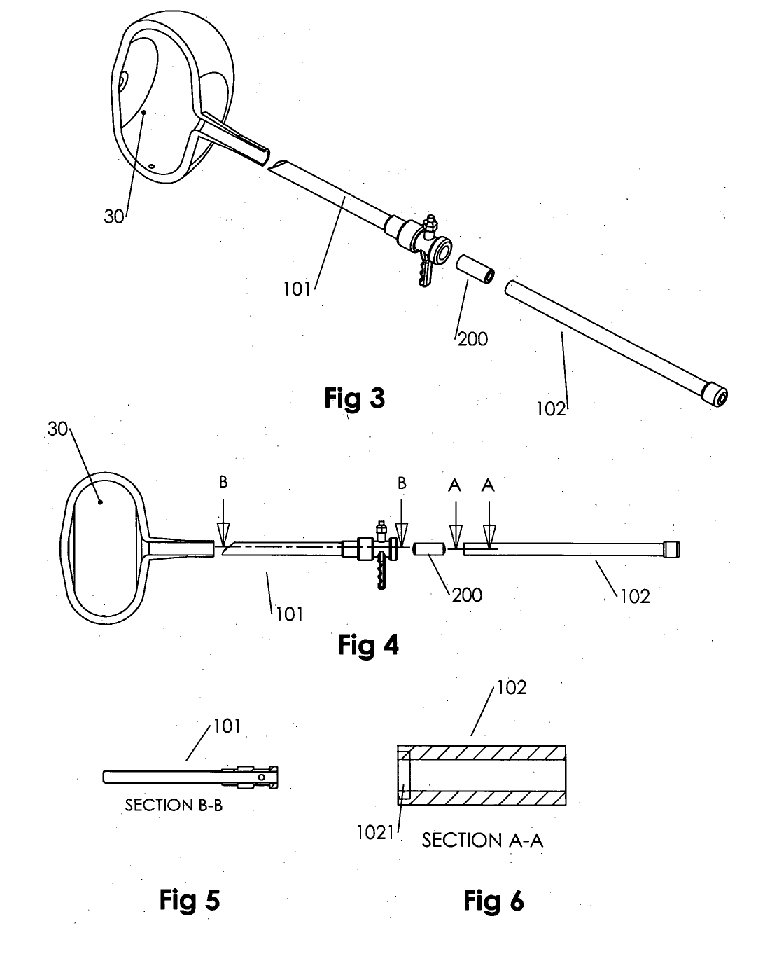

FIGS. 3 and 4 illustrate a urinary bladder of a person and portions of a device for inserting an implantable system into the urinary bladder according to an embodiment of the invention;

FIGS. 5 and 6 provide a cross sectional view of the device of FIGS. 3 and 4 according to an embodiment of the invention;

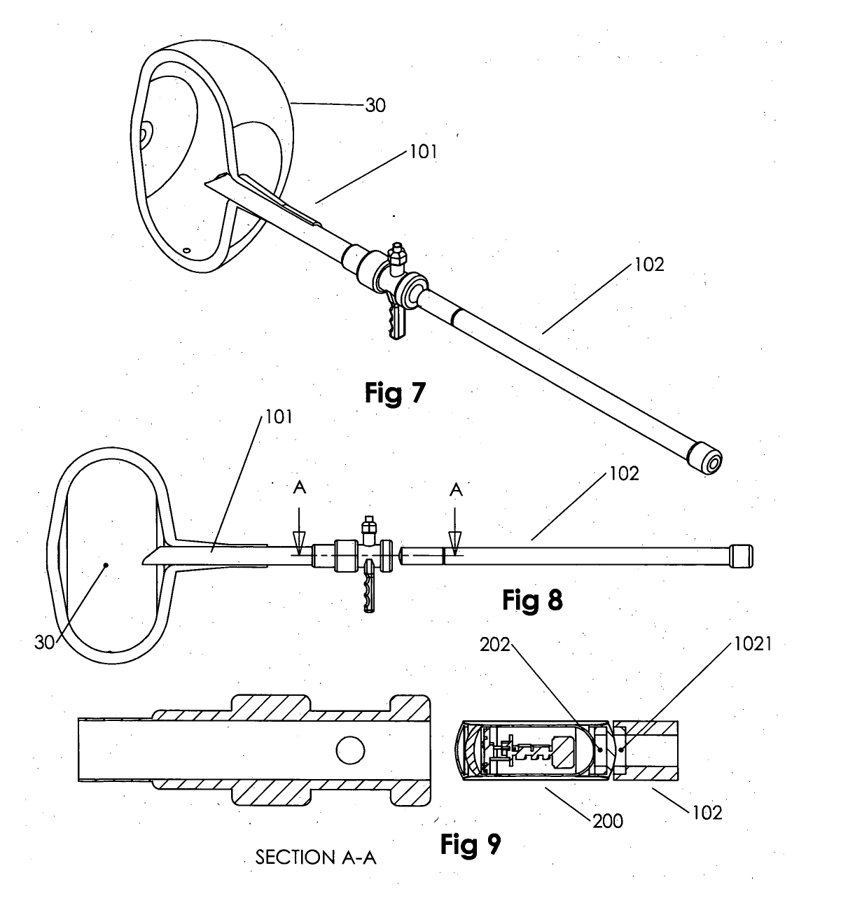

FIGS. 7 and 8 illustrate a urinary bladder of a person and portions of a device for inserting an implantable system into the urinary bladder according to an embodiment of the invention;

FIG. 9 provide a cross sectional view of the device, of FIGS. 7 and 8, of an imaging capsule and a magnet of an implantable system, according to an embodiment of the invention;

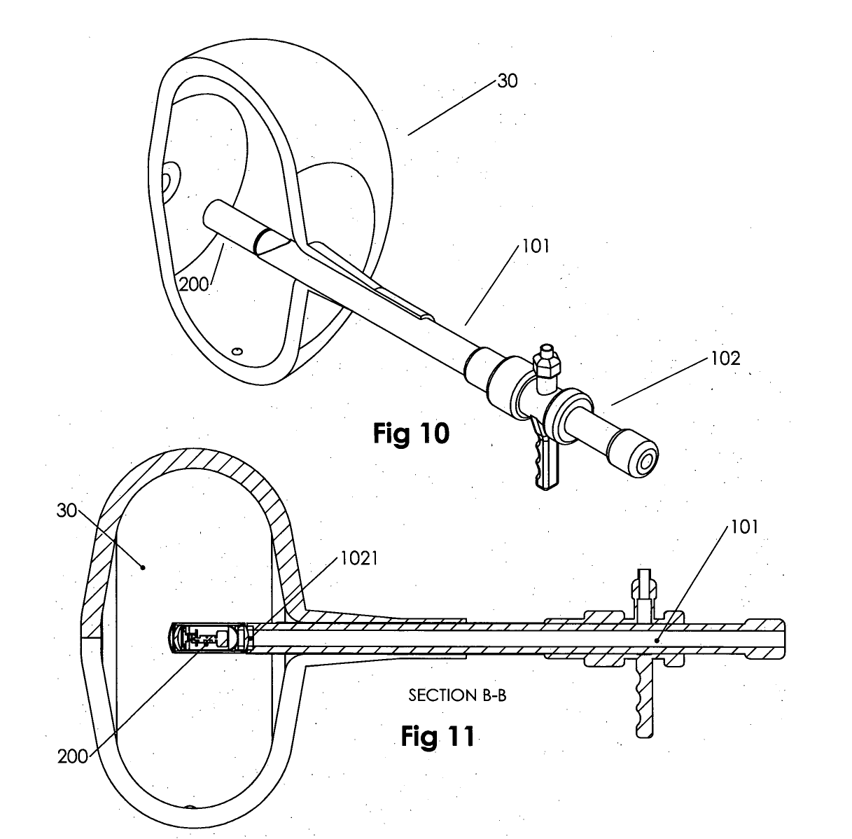

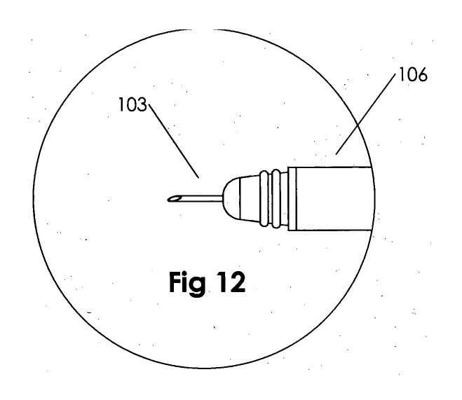

FIGS. 10 and 11 illustrate a urinary bladder of a person, an implantable system and portions of a device for inserting the implantable system into the urinary bladder according to an embodiment of the invention;

FIG. 12 provides an illustration, of a needle of the device of FIGS. 10 and 11 according to an embodiment of the invention;

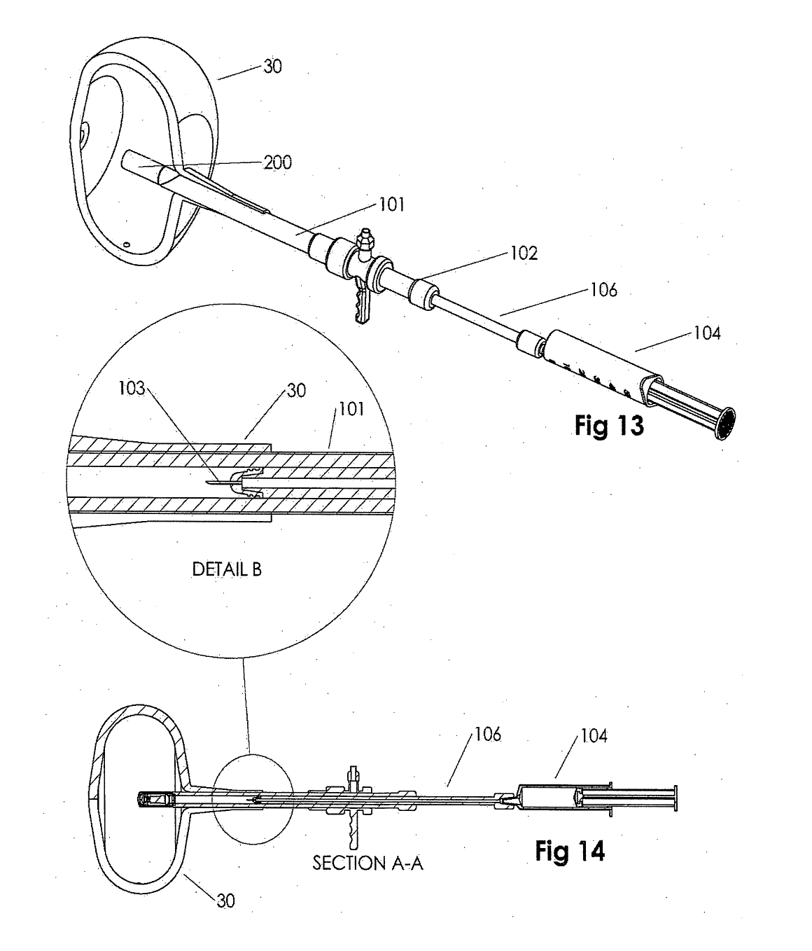

FIGS. 13 and 14 illustrate a urinary bladder of a person, an implantable system and portions of a device for inserting the implantable system into the urinary bladder according to an embodiment of the invention;

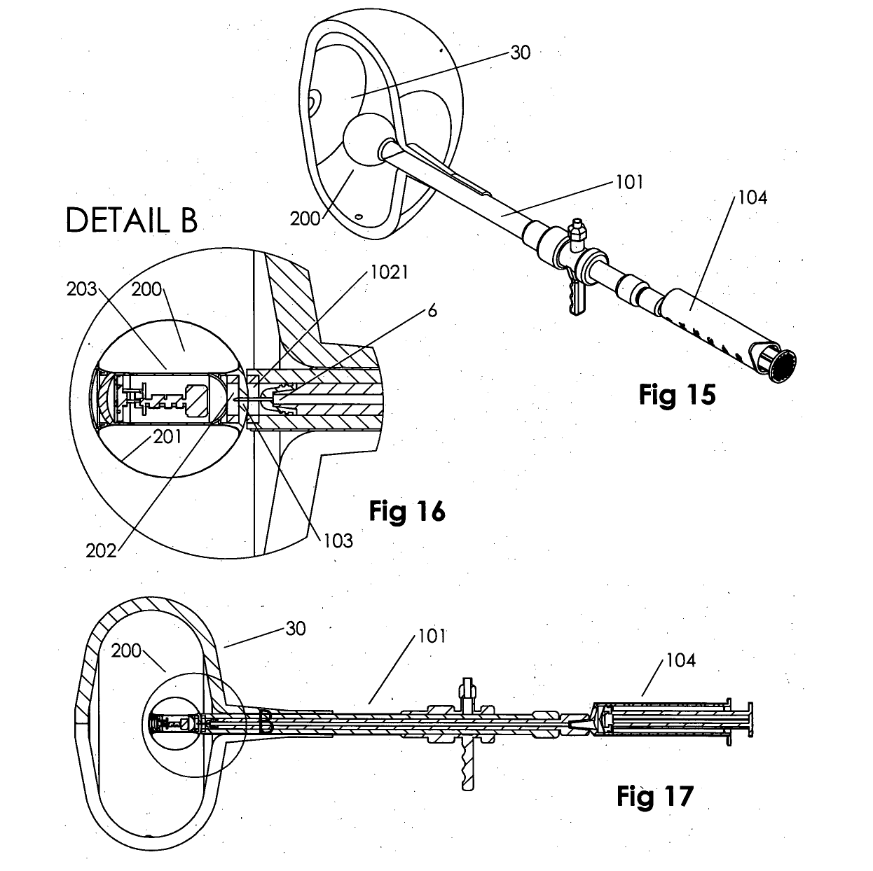

FIG. 15 illustrates a urinary bladder of a person, an implantable system and portions of a device for inserting the implantable system into the urinary bladder according to an embodiment of the invention;

FIG. 16 is a cross sectional view of an implantable system, of a needle, of a piston and of a cystoscope of the device for inserting the implantable system into the urinary bladder according to an embodiment of the invention;

FIG. 17 illustrate a urinary bladder of a person, an implantable system and portions of a device for inserting the implantable system into the urinary bladder according to an embodiment of the invention;

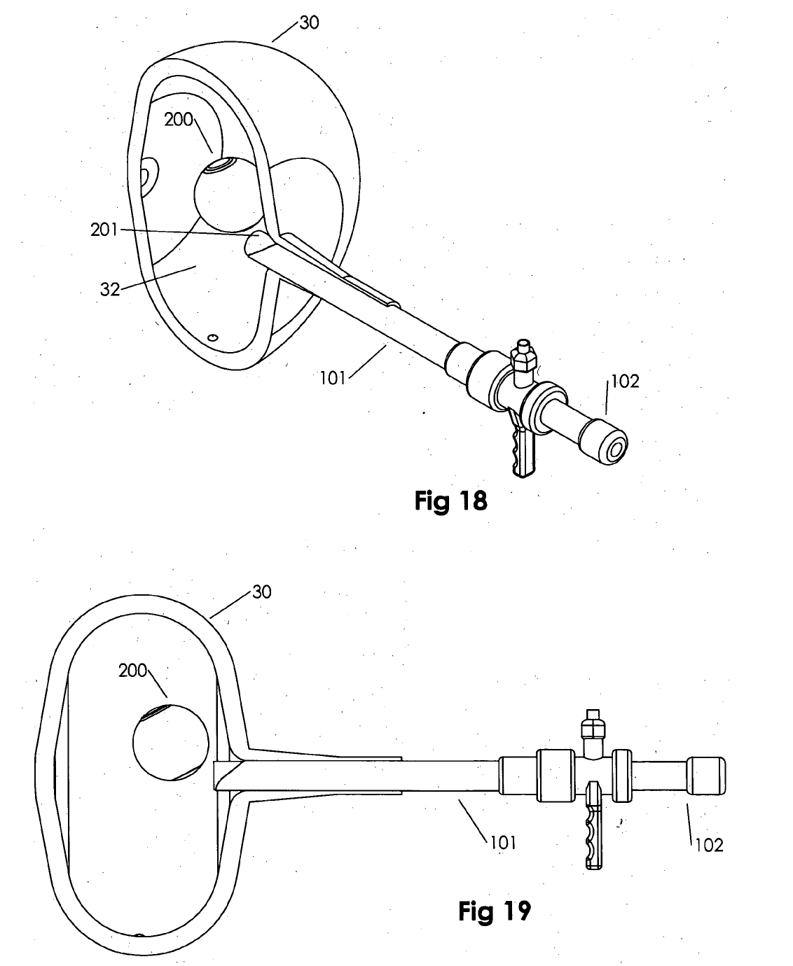

FIGS. 18 and 19 illustrate a urinary bladder of a person, an implantable system and portions of a device for inserting the implantable system into the urinary bladder after the implantable system is detached from the device and floats in the urine without contacting the walls of the urinary bladder, according to an embodiment of the invention;

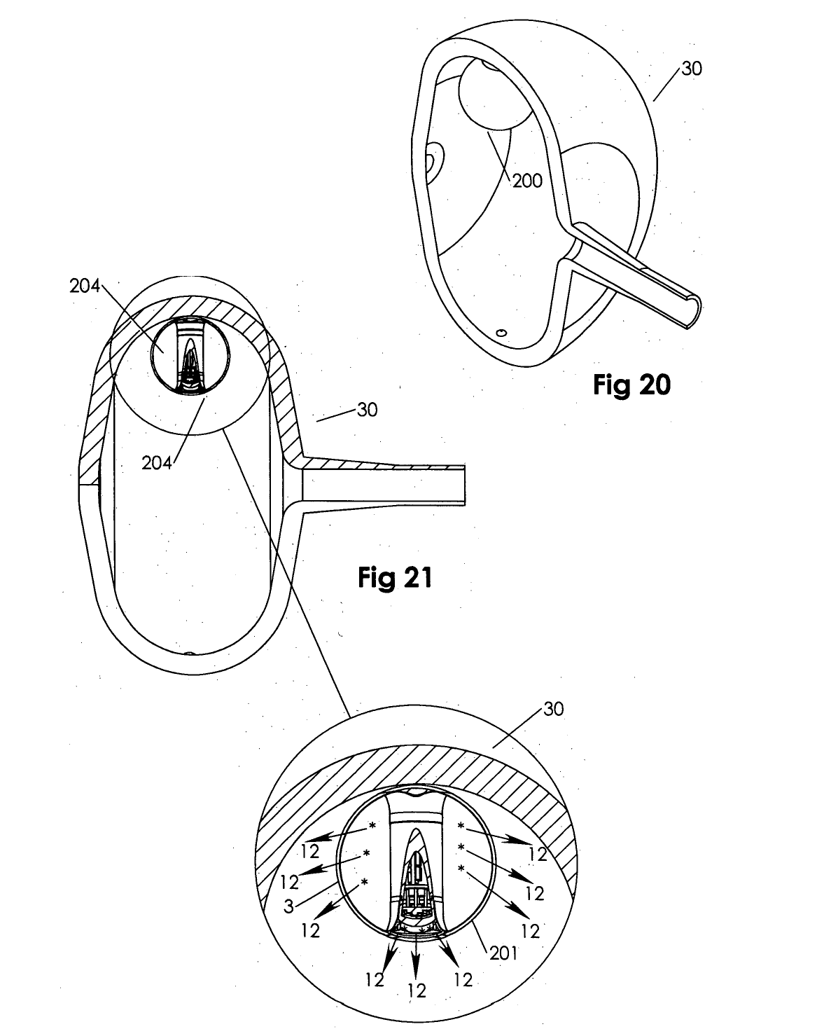

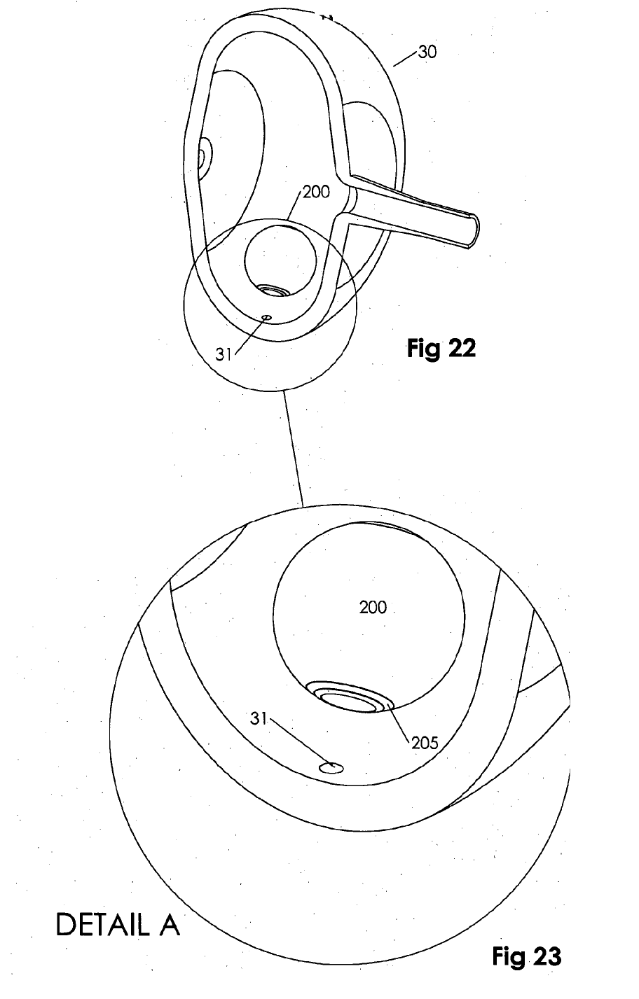

FIGS. 20, 21 and 22 illustrate a urinary bladder of a person and an implantable system wherein the implantable system contacts a wall of the urinary bladder, according to an embodiment of the invention;

FIGS. 22 and 23 illustrate a urinary bladder of a person and an implantable system wherein the implantable system faces a tumor developed on a wall of the urinary bladder, according to an embodiment of the invention;

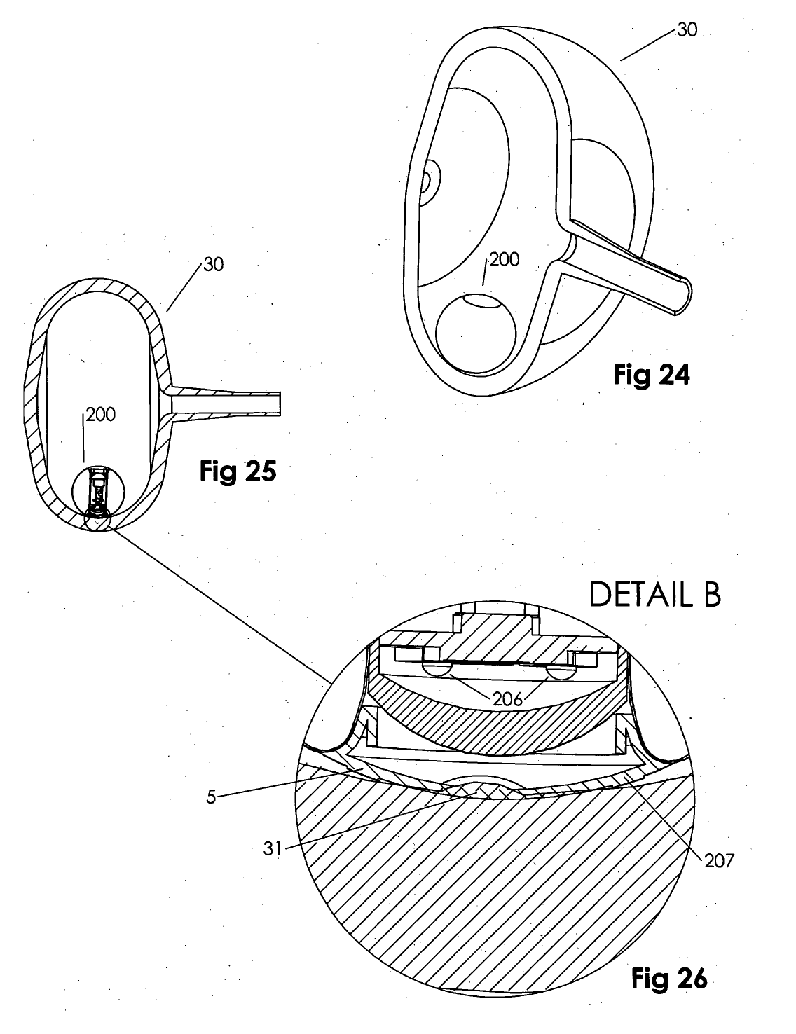

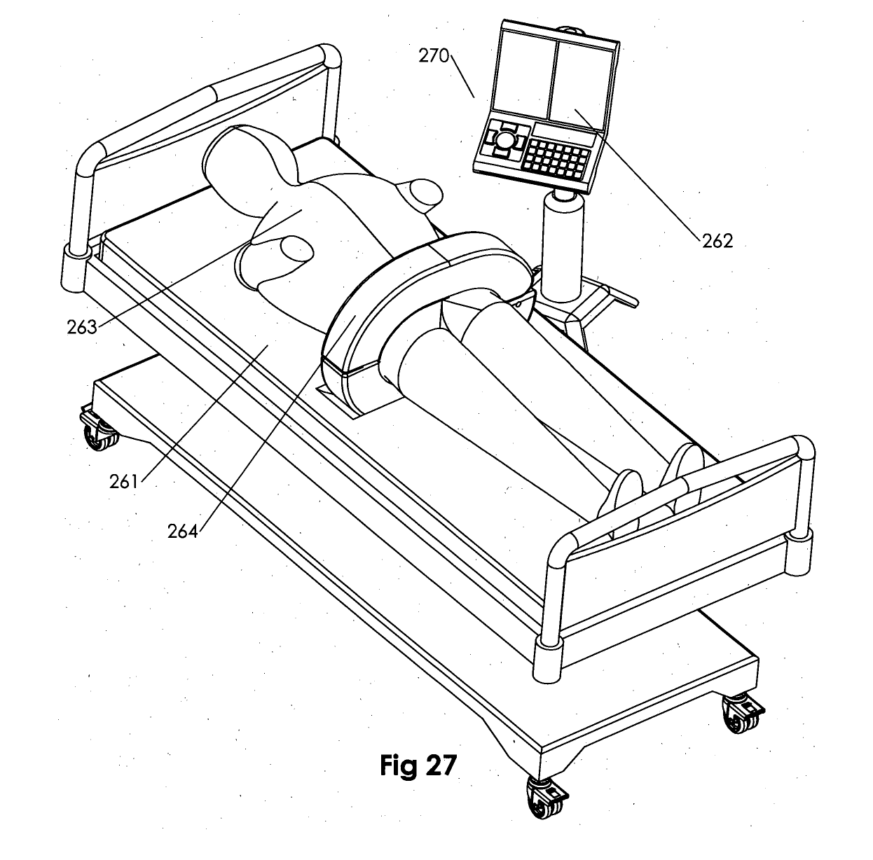

FIGS. 24, 25 and 26 illustrate a urinary bladder of a person and an implantable system wherein the implantable system touches a tumor developed on a wall of the urinary bladder, according to an embodiment of the invention;

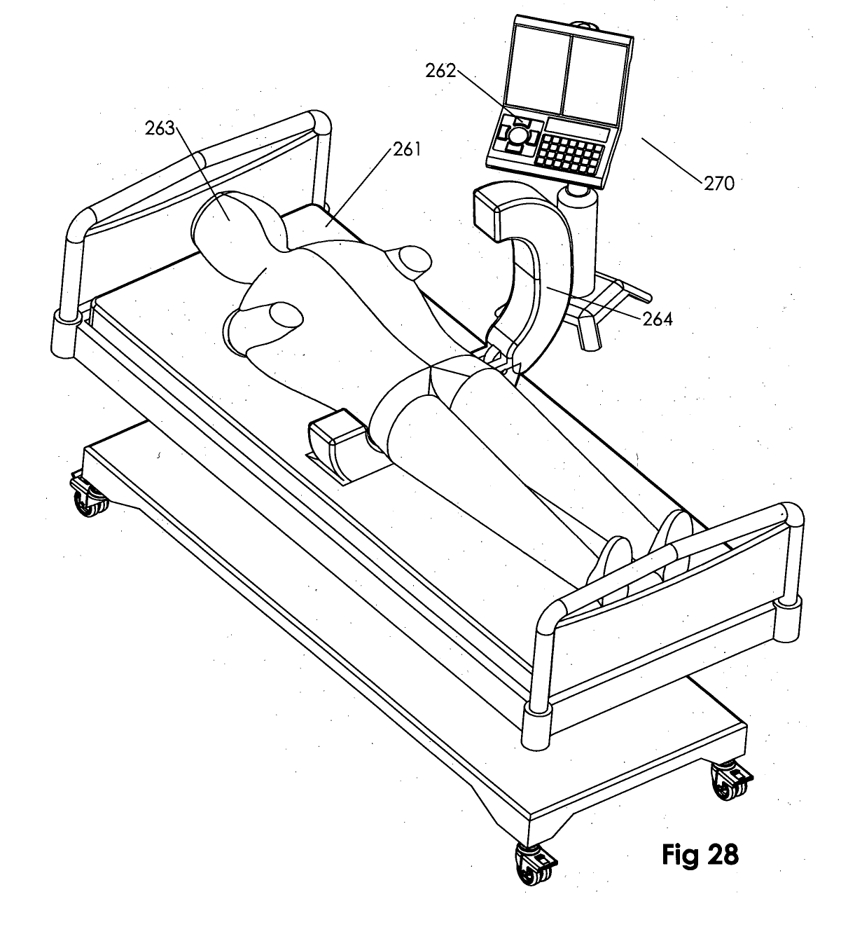

FIGS. 27 and 28 illustrate a person and a system for guiding the implantable system and for analyzing signals transmitted from the implantable system, according to an embodiment of the invention;

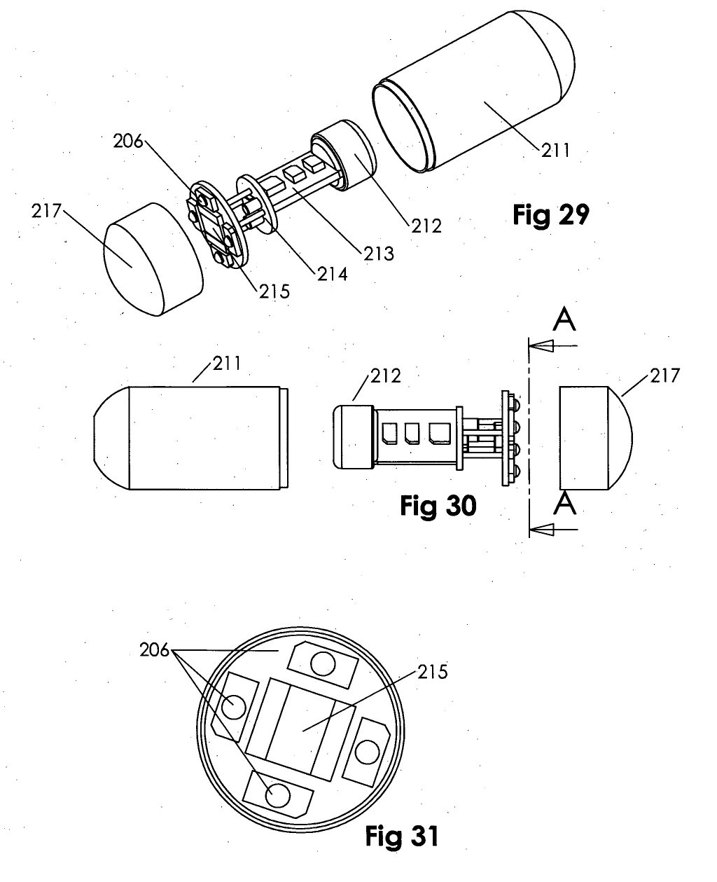

FIGS. 29 and 30 illustrate the implantable system, according to an embodiment of the invention;

FIG. 31 illustrates a lens and light emitting diodes of the implantable system, according to an embodiment of the invention;

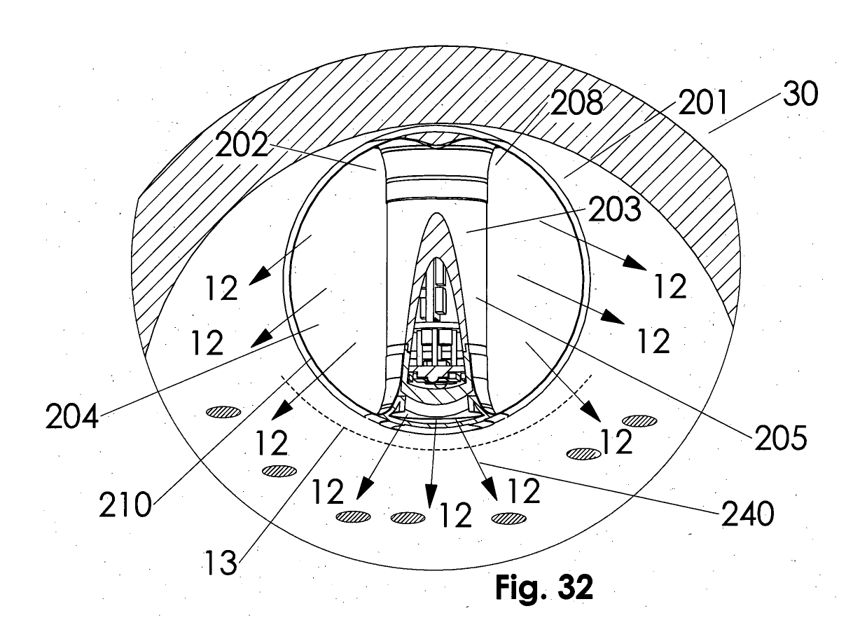

FIG. 32 illustrates a portion of a urinary bladder of a person, an implantable system inserted in the urinary bladder and also illustrates a flow of liquid from the implantable system, according to an embodiment of the invention;

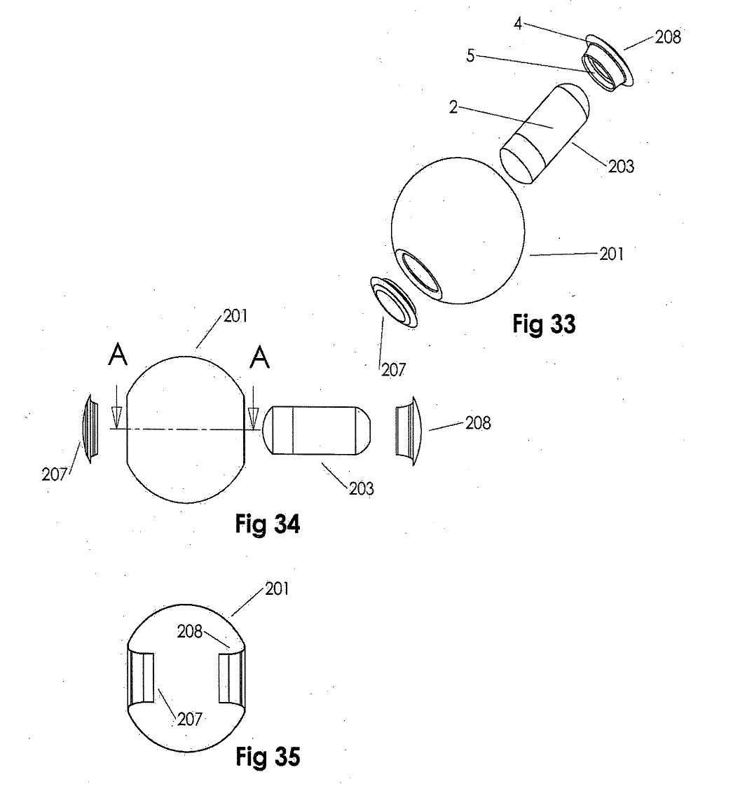

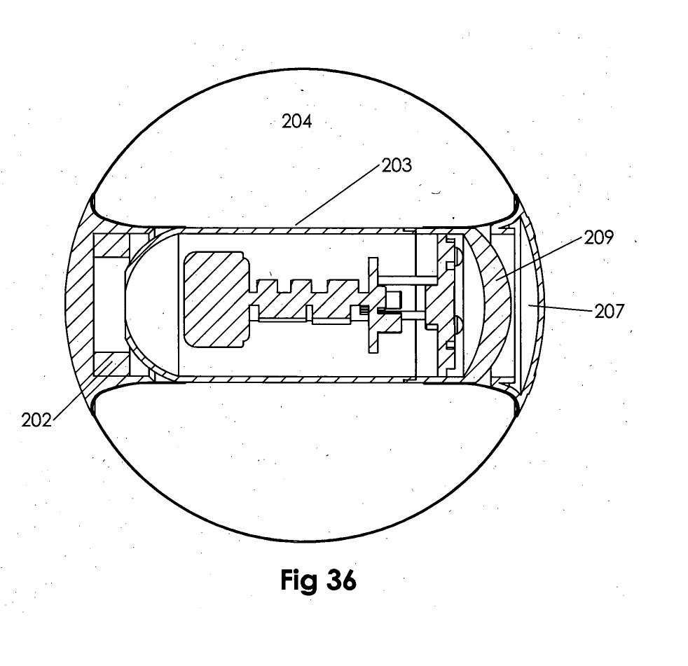

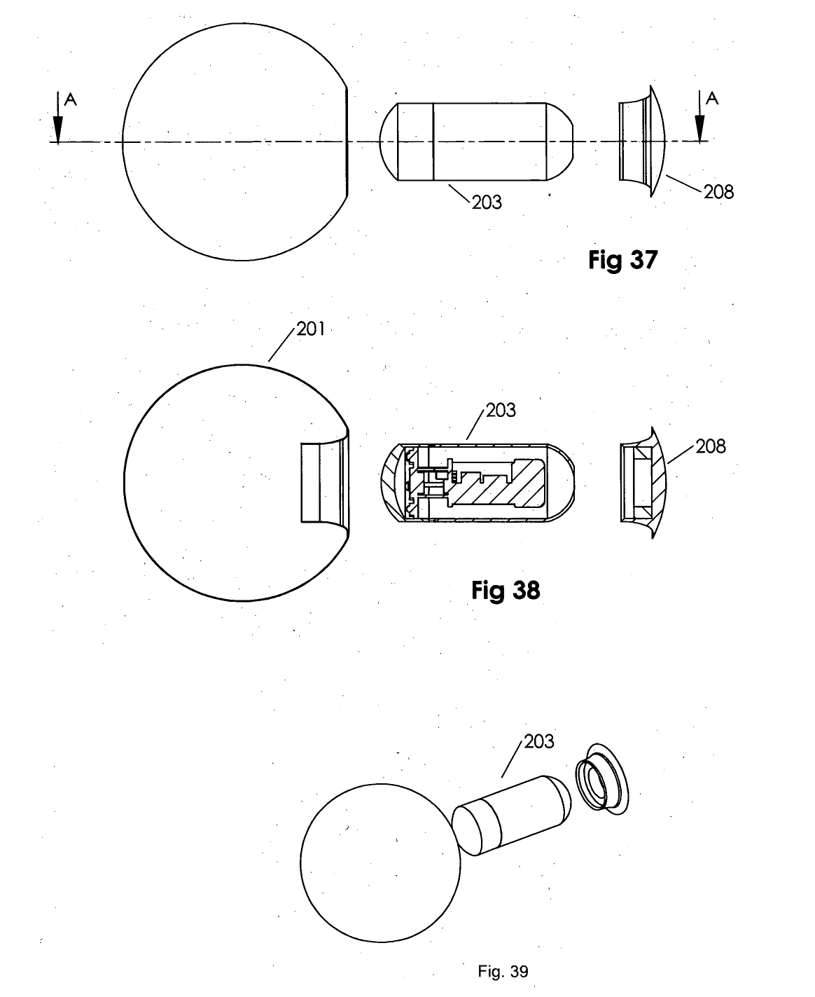

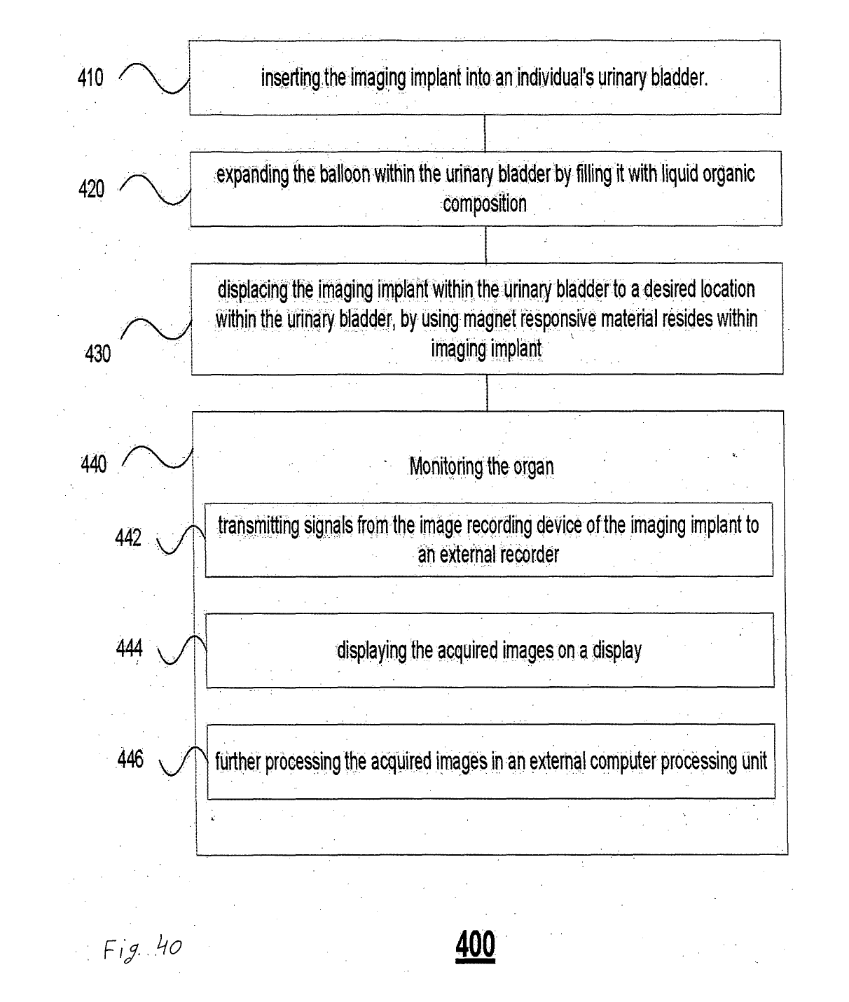

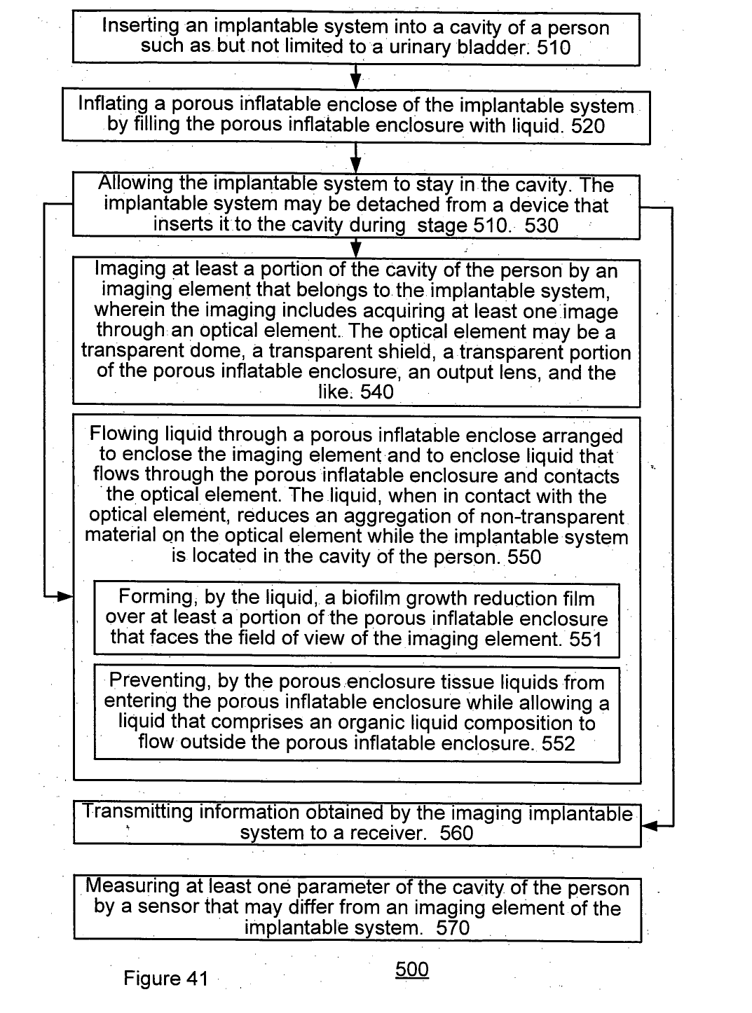

FIGS. 33, 34, 35, 36, 37, 38 and 39 illustrate portions of the implantable system according to an embodiment of the invention; and

FIGS. 40, 41 and 42 illustrate methods according to various embodiments of the invention.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The following example may refer to a urinary bladder. It is noted the systems, devices and method disclosed herein are not limited to operate, be inserted or image a urinary bladder and can be applied mutatis mutandis to other cavity of a person.

An implantable system is disclosed. It is implantable in the sense that it is relatively small (has a cross section that can be millimeteric) and can be made of materials that can be suitable for insertion into a body of a person.

### A Device for Inserting the Implantable System Into a Cavity of a Person

An implantable system can be inserted into a cavity of a person (such as the urinary bladder) by a device (also referred to as applicator) for inserting the implant into the cavity, and additionally or alternatively, for removing the implant from the cavity of the person.

The device can be fitted at an end thereof with a gripping device, and additionally or alternatively, by a magnet that attracts an implantable system to the device.

FIGS. 1 and 2 illustrate portions of device 100 for inserting an implantable system 200 into a cavity of a person according to an embodiment of the invention. FIGS. 3 and 4 illustrate urinary bladder 30 and portions of device 100 according to an embodiment of the invention. FIGS. 5 and 6 provide a cross sectional view of device 100 of FIGS. 3 and 4 according to an embodiment of the invention. FIGS. 7 and 8 illustrate urinary bladder 30 and device 100 according to an embodiment of the invention. FIG. 9 provides a cross sectional view of device 100 of FIGS. 7 and 8, of implantable system 200 and of magnets 202 and 1021, according to an embodiment of the invention. FIGS. 10 and 11 illustrate urinary bladder 30, implantable system 200 and portions of device 100 according to an embodiment of the invention. FIG. 12 provides an illustration of tip 106 of needle 106 of implantable system 200 of FIGS. 10 and 11 according to an embodiment of the invention. FIGS. 13 and 14 illustrate urinary bladder 30, implantable system 200 and portions of device 100 according to an embodiment of the invention. FIG. 15 illustrates urinary bladder 30, implantable system 200 and portions of device 100 according to an embodiment of the invention. FIG. 16 is a cross sectional view of implantable system 200, of needle 106, of piston 102 and of cystoscope body 101 of the device 100 according to an embodiment of the invention. FIG. 17 illustrates urinary bladder 30, implantable system 30 and portions of device 100 according to an embodiment of the invention. FIGS. 18 and 19 illustrate urinary bladder 30, implantable system 200 and portions of device 100 after the implantable system 200 is detached from device 100 and floats in the urine without contacting the walls of urinary bladder 30, according to an embodiment of the invention. FIGS. 20, 21 and 22 illustrate urinary bladder 30 and implantable system 200 wherein implantable system 200 contacts a wall of urinary bladder 30, according to an embodiment of the invention. FIGS. 22 and 23 illustrate urinary bladder 30 and implantable system 200 wherein the implantable system 200 faces tumor 31 developed on a wall of urinary bladder 30, according to an embodiment of the invention. FIGS. 24, 25 and 26 illustrate urinary bladder 30 and implantable system 200 wherein implantable system 200 touches tumor 31 that developed on a wall of urinary bladder 30, according to an embodiment of the invention.

Referring to FIGS. 1-4, device 100 includes a hollow element (such as cystoscope body 101), a piston 102 shaped to be inserted in a cavity of the cystoscope body 101 and has a magnet (not shown), a needle 106 for injection of liquid into implantable system 200, and syringe 104 for filling the implantable system 220 with the liquid.

The output of syringe 104 is detachably connected to needle 106 that ends with tip 103. Needle 106 is long and may be inserted into an inner cavity of piston 102.

Piston 102 and implantable system 200 are shaped so as to be inserted into an inner space of cystoscope body 101. Cystoscope body 10 also may include a handle that can be held by a medical personal during the insertion of the implantable system into urinary bladder 30.

Piston 102 ends with a magnet (1021 of FIG. 6) that attracts a magnet (denoted 202 in FIG. 32) of implantable system 200. It is noted that piston 102 may be connected to implantable system 200 by other (detachable) means.

The insertion of implantable system 200 in urinary bladder 30 may include the following stages:

(i) Detachably connecting Implantable system 200 to one end of piston 102. At this stage the implantable system 200 can be in a non-inflated mode. FIGS. 2 and 4 illustrate implantable system 200 in proximity to piston 102—before being connected to the piston 102.

(ii) Inserting cystoscope body 101 into urinary bladder 30.

(iii) Inserting implantable system 200 and piston 102 into the cystoscope body 101. FIGS. 7 and 8 illustrate the piston 102 that is barely inserted in the cystoscope body 101.

(iv) Forcing piston 102 to enter cystoscope body 101 until implantable system 200 emerges from cystoscope body 101 and enters urinary bladder 30. FIGS. 10 and 11 illustrates implantable system 200 after this stage—it exits cystoscope body 101 and is held by magnetic attraction forces to magnet 1021 of piston 102.

(v) Inserting needle 106 (after being connected to syringe 104) into piston 102 until the needle 106 contacts (and even penetrates) implantable system 200. FIGS. 13 and 14 illustrate an intermediate position in which needle 106 enters the piston 102 but does not reach implantable system 200.

(vi) Filling, by syringe 104 and needle 106 (and especially tip 103 of needle 106) liquid into implantable system 200. FIGS. 15, 16 and 17 illustrate the end of this stage—the syringe is empty and the liquid fully inflates implantable system 200.

(v) Detaching implantable system 200 from piston 102 and maintaining implantable system 200 within urinary bladder 30. Implantable system 200 can float within urinary bladder 30—as illustrated in FIGS. 18 and 19. The detachment can be assisted by applying mechanical forces on piston 102 or on implantable system 200. Additionally or alternatively, the detachment can be assisted by the inflating of the implantable system and especially by a tendency of implantable system 200 to float once fully (or almost fully) inflated.

It is noted that the mentioned above stages are not mandatory and that their order can be changed. For example—implantable system 200 can be inserted in the cystoscope before that latter is inserted into urinary bladder 30.

Implantable system 200 may be extracted from urinary bladder 30 by a reverse process.

It is noted that implantable system 200 can be refilled with liquid by inserting device 100 into urinary bladder 30, forcing (for example—by applying magnetic fields) implantable system 200 to contact device 100 and injecting liquid via one way valve 220 of implantable system 200.

FIGS. 27 and 28 illustrate person 263 and system 270 for guiding the implantable system'and for analyzing signals transmitted from the implantable system, according to an embodiment of the invention.

System 270 includes a magnetic field inducer 264 that surrounds an area that in turn includes the urinary bladder and its surroundings of person 263 and can generate one or more magnetic fields of desired amplitude and direction in order to guide the implantable system in the urinary bladder. Implantable system 200 may wirelessly transmit image information to monitor 262 (directly or via an intermediate device) that can process the image information and display images or other data on a display of monitor 262.

FIG. 27 illustrates magnetic field inducer 264 when in a closed position in which is surrounds person 263 (that lies on bed 261) while FIG. 28 illustrates magnetic field inducer 264 when in an open position.

### The Implantable System

According to an embodiment of the invention an implantable system for insertion into a cavity of a person is provided. The implantable system includes: (i) an imaging element capable of acquiring an image of at least a portion of the cavity through an optical element; and (ii) a porous inflatable enclosure arranged to enclose the imaging element and to enclose liquid that flows through the porous inflatable enclosure and contacts the optical element. The liquid, when in contact with the optical element, reduces an aggregation of non-transparent material (such as scar tissue, biofilm) on the optical element while the implantable system is located in the cavity of the person.

According to an embodiment of the invention an implantable system is provided. The system includes: (i) an imaging element; and (ii) a porous inflatable enclosure arranged to enclose the imaging element and to enclose liquid that flows through the porous inflatable enclosure, wherein the flow of liquid reduces a growth of biofilm or scar tissue on the implantable system, when the implantable system is located in the cavity of the person.

The imaging element can be an imaging capsule that is enclosed within the porous inflatable enclosure. The flow of liquid may protect the external surface of the porous inflatable enclosure from biofilm growth. The fluid provides a clear viewing path from the imaging capsule to a target surface of a tissue to be imaged, for desired periods, and in particular extended periods. The liquid may be a liquid organic composition.

The implantable system of the present invention may be left in the body for prolonged periods of time without growing biofilm, encrusting or causing bacterial infections and its location can be determined (after being inserted in the body) by using varying magnetic fields such as hand held magnet. The implantable system structure may be fabricated to be comfortable for the patient and does not interfere with the subject routine activities.

The applicants unexpectedly discovered that permeation (for example—continuous) of a organic liquid compositions through a polymer membrane made porous inflatable enclosure minimizes the growth of biofilm on the membrane when immersed in body liquids such as urine.

The continuous permeation of the liquid organic compound serves two purposes: first, it minimizes accumulation of protein from the urine on the enclosure surface and second, it interferes with the bacteria adhesion process on the enclosure wall. The protein layer is considered a pre-condition which promotes bacterial attachment onto surface in continuous contact within the urine.

FIG. 32 illustrates implantable system 200 that floats in urine bladder 30, according to an embodiment of the invention.

Implantable system 200 includes an imaging element such as imaging capsule 203 that is enclosed in a porous inflatable enclosure such as balloon 201. The porous inflatable enclosure is arranged to enclose imaging capsule 203 and to enclose liquid 215 that flows through the porous inflatable enclosure 201. The flow of liquid 215 (illustrates by arrows 12 that extend from the porous inflatable enclosure towards the urine bladder 30) prevents a biofilm from growing on implantable system 200 and especially from growing over an optical element such as transparent dome 207 through which imaging capsule 203 images urinate bladder 30.

The fluid prevents (or at least substantially reduces) non-transparent material from aggregating on optical dome 207 and from obscuring the field of view of imaging capsule 203, when implantable system 200 is located in the cavity of the person. Without such flow of liquid the visibility of imaging capsule 203 may be limited or even reduced to zero by such obstruction.

Balloon 201 may be made of polymer material and may be filled with a transparent liquid organic composition 215, whose density at 37° C. is between 0.65 and 0.9 gram per cubic cm.

Balloon 201 may be filled after being inserted in the cavity or alternatively, filled and compressed prior to insertion of the device into the body cavity and then allowed to expand after insertion into the body cavity. Balloon 201 can be partially filled with the fluid before being inserted into the body and then be fully filled after being inserted.

Balloon 201 may shield imaging capsule 203 from the body fluids.

Balloon 201 or imaging capsule 203 can include a magnetizable element 202 that may assist in the positioning of balloon 201 within the body (after being inserted). FIG. 32 illustrates magnetic ring 202 that surrounds imaging capsule 203. It is used for attracting implantable system 202 during a procedure of retrieval from implantable system 202.

Balloon 201 can be caught by (or otherwise be connectible to or attracted to) retrieval means for retrieval of imaging capsule 203. Balloon 201 may prevent imaging capsule 203 from adhering to the internal surface of the tissue or organ (said prevention may be assisted by the flow of fluid through balloon 201).

Additionally or alternatively, the balloon 201 may prevent unwanted exit of implantable device 200 from the cavity (for example—from urinary bladder 30) by being larger than the output orifice of that organ—after balloon 201 is inflated.

Balloon 201 (or at least a portion through which imaging capsule 203 images the tissue) is optically transparent and can include one way valve 208 for filling balloon 201 with the liquid organic composition 215. It is noted that other manes for inserting the fluid can be used. The one way value 208 and the transparent dome 207 are also illustrated in FIGS. 33-39.

After inserting implantable system 200 into a urinary bladder 30, it is inflated to the proper pressure (or desired volume) with liquid such as liquid organic composition 215 through one-way valve 208.

The wall of balloon 201 may be manufactured in such a way that it is porous and such that liquid organic composition 215 can slowly seeps through the porous wall, generating a thin and relatively uniform layer 13 of liquid organic composition 215 over the outer surface of balloon 201 (or at least transparent optical dome 207) or other elements through which the imaging capsule 203 images the tissue. The seeping is illustrated by arrows 12. The seeping can be done by osmosis and the holes in the balloon 201 can be of sub-millimeteric and even microscopic dimensions. Multiple holes can surround or otherwise be located near transparent dome 207 so that once the liquid organic composition flows through them it covers transparent dome 207 with a transparent film of the liquid organic composition.

Each one of the slow seeping rate, the viscosity of the liquid organic composition 205, the texture of the external surface of balloon 201 and the fact that the balloon is at least partially surrounded by urine or other body liquids assists in the formation of the film.

The seeping rate of liquid organic composition 215 during the retention of implantable system 200 within urinary bladder 30 may be optimized such that only a relative small fraction of liquid organic compound 215 is lost after the designated retention period (prolonged period) of implantable system 200 within urinary bladder 30. The wall porosity of balloon 201 is also optimized such that the seeping rate of organic liquid composition 215 is sufficient for maintaining a thin and uniform layer 13 on the external surface of balloon 201. The material from which balloon 201 is made as well the dimensions (thickness) of balloon 201 affect the seeping rate. Thicker walls may result in a lower seeping rate. The seeping rate may be slow enough to allow the liquid organic compound 215 to seep during a prolonged period of weeks, months and even more. A 200 micron thick wall allowed 20% of the liquid organic compound 215 to seep during a period of two years.

The seeping rate can be responsive to a pressure difference between the liquid organic composition 215 within the implantable system 200 and a body liquid (such as urine) that at least partially surrounds the implantable system 200.

The density of liquid organic composition 215 may be determined such that implantable system 200 slightly floats within the urine which fills urinary bladder 30.

Balloon 201 (especially the balloon wall) may be manufactured from a hydrophobic membrane operable to be wetted by liquid organic composition 215 and to permit diffusion through balloon 201 and in contrast to prevent water diffusion through balloon 201.

In other aspects, balloon 201 may manufactured so that the diffusion rate of the liquid organic composition 215 is sufficient to renew layer 13 on the external surface of balloon 201, in cases that the internal surface (wall) of urinary bladder 30 wipes a portion of layer 13 by contact with the external wall of balloon 201—as illustrated in FIGS. 20, 21 and 22.

According to another embodiment, liquid organic composition 215 which fills balloon 210 includes encapsulated gas bubbles which assist implantable system 200 to float in urinary bladder 30.

Conveniently, the external surface of balloon 201 may include a texture which assists in continuously maintaining layer 13 of liquid organic composition 215 on the outer wall of balloon 201.

In certain embodiments, liquid organic composition 215 is a mineral oil. In further embodiment, a significant fraction of liquid organic composition 215 is composed of linear aliphatic molecules with carbon atom count between 8 and 25. In yet another embodiment, liquid organic composition 215 is inert and thus does not induce any interaction when released within urinary bladder 30.

Balloon 201 can be filled with a liquid that is either a drug or includes a drug.

Implantable system 200 may include at least one sensor that differs from imaging capsule 203 for measuring at least one parameter of the cavity. It can include a pressure sensor for measuring the pressure of the liquid in the cavity.

FIGS. 29, 30 and 31 illustrate imaging capsule 203 and portions of the imaging capsule 203 according to an embodiment of the invention.

Imaging capsule 203 may includes image acquisition related elements, such as: (i) an electronic image recording device; (ii) an optical imaging assembly, including an image sensor (e.g. CMOS or CCD), for an image acquisition of a cell, tissue or organ and storing thereof in a suitable image recording device.

Imaging capsule 203 may capture images at any frame rate and at any exposure time, meaning the images may include still images or video images.

Imaging capsule 203 may include a focusing element, device or software based.

Imaging capsule 203 may include one or more lenses such as a compound lens, liquid lens, prismatic lens, a sensor lens and the like. The imaging lens may be a microscope lens and may be equipped with a spectral filter which may cover at least a portion of the image extent.

Imaging capsule 203 may include an illumination source for illuminating the cell, tissue or organ imaged onto said image sensor, such as but not limited to a light emitting diode (LED), an ultraviolet light source, an infrared light source, an incandescent light source or any combination thereof. The spectral distribution, intensity and spatial intensity distribution provided by the illumination source may be predetermined, to comply with the environment which it is meant to illuminate and with the image sensor which may need the illumination in order to capture useful image or spatial information. For example, a near-IR illumination source may be used with an image sensor that is adapted to capture images at the near-IR spectrum. FIGS. 29-31 illustrates four spaced apart LEDs 206. These LEDs surround a rectangular lens 215 of the imaging capsule 203.

Imaging capsule 203 may also include electric power source (212 that may include any type of rechargeable batteries that are known in the art, such as, but not limited to, Li-Ion, Ni-Me, Ni-Cd and the like or electrolyte capacitor. The power source may be recharged from an external source, such as, magnetic induction, electrical induction, chemical charging and the like. The power source may also be recharged by an adaptable-wired connection to an external power source.

Imaging capsule 203 also include (214, a processor for processing the images obtained by the wireless transmitter for transmitting acquired images and/or electronic signals to an external receiver.

According to some embodiments, images captured by the image sensor (215 may be viewed on-line in real time by a user, such as a health care provider (Physician, technician, nurse and the like).

To this aim, the wireless-transmitter may transmit the acquired images to the system such as system 267 of FIGS. 27 and 28. The wireless transmitter 213 can transmit the image information to a relay, to a long range transceiver, to a mobile phone, to a remote system, or to a system that is proximate to the person. The wireless transmitter 213 can be proximate to the person and even attached to the person.

An external receiver that receives the image information from the wireless transmitter may be operated by a user, such as a health care provider and may further allow the user to view the acquired image data.

According to further examples, the image data acquired by the image sensor is not transmitted to an external source but rather stored in the implantable system. The stored data may be retrieved as is or processed by a user at a later time.

According to an embodiment of the invention, implantable system 200 is a portion of a catheter, a stent or other devices intended to be inserted into the urinary bladder 30.

According to an embodiment of the invention implantable system 200 can include a heater such as a heating ring (denoted 205 in FIG. 23) that can disperse heat. The heater can be attached to the balloon 210 after the balloon 201 is formed or during the manufacturing process of the balloon 201. For example, by using ablation, the heating ring can be, heated (for example-heated to 55 degrees for 3 minutes) and once heated is attached to balloon 201. Heating ring 205 can be connected to a heater (not shown) located inside balloon 201.

Balloon 201 is illustrated as having a single wall but this is not necessarily so. Balloon 201 may include many layers and may include multiple inner compartments.

### Method for Imaging

FIG. 40 illustrates a method 400 for implanting an implantable system and monitoring the interior of an individual's urinary bladder.

Method 400 starts with stage 410 of inserting an implantable system into an individual's urinary bladder. Conveniently the insertion is through a urethra, using a handset. Stage 410 includes releasing the implantable system into the urinary bladder and extracting the handset from the urethra. The implantable system may also be inserted into a urinary bladder using an endoscope, catheters, laparoscopic surgery, and the like.

Stage 410 is followed by stage 420 of expanding a porous inflatable enclosure such as a balloon within the urinary bladder by filling it with liquid such as a liquid organic composition.

Stage 420 is followed by stage 430 of displacing the implantable system within the urinary bladder to a desired location within the urinary bladder, by using a hand-held magnet for manipulating a magnetizable element of the implantable system.

Stage 430 is followed by stage 440 of monitoring. Stage 440 can include at least one of the following stages: stage 442 of transmitting signals from the image recording device of the implantable system to an external recorder. The external recorder can be coupled to a computer storage; stage 444 of displaying the acquired images on a display; and stage 446 of further processing the acquired images in an external computer processing unit. Stage 446, conveniently includes storing the processed images and displaying the processed images.

The seeping of liquid organic composition 215 takes place from. stage 420 at least until the implant 202 is retrieved from the urinary bladder.

The examples set forth above relate to the urinary bladder but the invention can be used for imaging of any cells, tissue or organ of said subject without interfering with the implant's image recording capability and without exposing said internal organ to danger of bacterial contamination.

FIG. 41 illustrates method 500 according to an embodiment of the invention.

Method 500 starts by stage 510 of inserting an implantable system into a cavity of a person such as but not limited to a urinary bladder.

Stage 510 may be followed by stage 520 of inflating a porous inflatable enclose of the implantable system by filling the porous inflatable enclosure with liquid.

Stage 520 may include inserting the liquid in the porous inflatable enclosure through a one way valve.

Stage 520 includes inserting the liquid in the porous inflatable enclosure and thereby expanding the porous inflatable enclosure, after the implantable system is inserted to the cavity of a person. It is noted that at least a part of the filling process can occur before the insertion or before the insertion is completed. Thus, method 500 can include inserting the liquid in the porous inflatable enclosure and thereby expanding the porous enclosure before the implantable system is inserted to the cavity.

Stage 520 may be followed by stages 530, 540 and 550.

Stage 530 includes allowing the implantable system to stay in the cavity. The implantable system may be detached from a device that inserts it to the cavity during stage 510.

Stage 530 may include positioning the porous inflatable enclosure in a urinary bladder without sealing the urinary bladder.

Stage 530 may include positioning the implantable system in the cavity by applying a magnetic field on a magnetizable element of the implantable system.

Stage 530 may include allowing the implantable system to float in urine.

Stage 530 can include guiding (including orienting) the implantable system to desired locations and/or orientations in order to obtain images of desired portions of the cavity.

Stage 540 includes imaging at least a portion of the cavity of the person by an imaging element that belongs to the implantable system, wherein the imaging includes acquiring at least one image through an optical element. The optical element may be a transparent dome, a transparent shield, a transparent portion of the porous inflatable enclosure, an output lens, and the like.

Stage 550 includes flowing fluid through a porous inflatable enclose arranged to enclose the imaging element and to enclose liquid that flows through the porous inflatable enclosure and contacts the optical element. The liquid, when in contact with the optical element, reduces an aggregation of non-transparent material on the optical element while the implantable system is located in the cavity of the person.

Stage 550 may include flowing the liquid during a prolonged period through holes of the porous inflatable enclosure that are sized to facilitate the flowing during the prolonged period.

Stage 550 may include stage 551 of forming, by the liquid, a biofilm attachment reduction film over at least a portion of the porous inflatable enclosure that faces the field of view of the imaging element.

Stage 551 may include maintaining the film during a prolonged period.

The prolonged period may exceeds one or more days, one or more weeks, one or more months and even one or more years. The prolonged period may be long is relation to a period required for biofilm to grow, at an absence of the film, to an amount that prevents the imaging element to image the tissue.

Stage 551 may include maintaining a film that is developed over the entire porous inflatable enclosure.

Stage 550 may include stage 552 of preventing, by the porous enclosure tissue liquids from entering the porous inflatable enclosure while allowing a liquid that comprises an organic liquid composition to flow outside the porous inflatable enclosure.

The liquid may be an organic liquid composition. The liquid may be an organic liquid composition that is at least partially transparent and has a density, at 35 degrees Celsius that ranges between 0.65 and 0.9 gram per cubic centimeter. The liquid may be an organic liquid composition that is at least partially transparent and is inert. The liquid may be a mineral oil. The liquid may be an organic liquid composition that comprises linear aliphatic molecules with a carbon atom count that ranges between 8 and 24. The liquid may be a liquid organic composition that encapsulates gas bubbles. The liquid may be a medical drug or include a medical drug.

The porous inflatable enclosure may be made of a hydrophobic membrane. The holes of the porous inflatable enclosure may be evenly (or unevenly) spaced over the porous inflatable enclosure. At least a portion of an external surface of the porous inflatable enclosure may have a texture that is shaped to assist in the generation of the film.

Method 500 may also include stage 560 of transmitting information obtained by the imaging implantable system to a receiver. Stage 560 may include transmitting the image information to a receiver located outside the person, to a transceiver implanted in the person and the like. Stage 560 may include transmitting still images or video acquired by the imaging element of the implantable system.

Method 500 may also include stage 570 of measuring at least one parameter of the cavity of the person by a sensor that may differ from an imaging element of the implantable system. Stage 570 may include measuring an intra-vesicle pressure of the person.

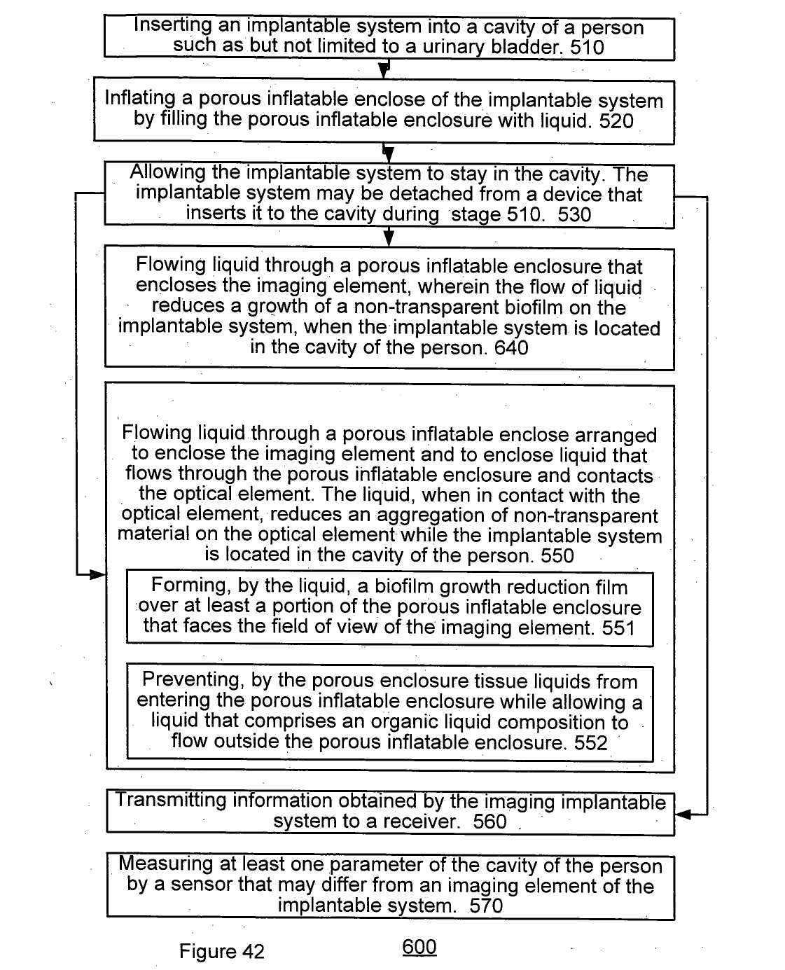

FIG. 42 illustrates method 600 according to an embodiment of the invention.

FIG. 600 includes stage 640 instead of stage 540. The implantable system inserted during stage 510 has an imaging element that has a field of view. Stage 640 includes flowing liquid through a porous inflatable enclosure that encloses the imaging element, wherein the flow of liquid reduces an attachment (for example by growth or merely by being stuck to) of a non-transparent biofilm on the implantable system, when the implantable system is located in the cavity of the person.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. An implantable system for insertion into a cavity of a person, comprising:
an imaging element capable of acquiring an image of at least a portion of the cavity through an optical element; and
a porous inflatable enclosure arranged to enclose the imaging element and to enclose liquid that flows through the porous inflatable enclosure and contacts the optical element, wherein the liquid, when in contact with the optical element, reduces an aggregation of non-transparent material on the optical element while the implantable system is located in the cavity of the person.

2. The implantable system according to claim 1, wherein holes of the porous inflatable enclosure are sized to facilitate a flow of the liquid during a prolonged period.

3. The implantable system according to claim 2 wherein the porous inflatable enclosure is shaped to allow the liquid to form a biofilm attachment reduction film over at least a portion of the implantable system that faces the field of view of the imaging element.

4. The implantable system according to claim 3, wherein the implantable system is arranged to maintain the film during a prolonged period.

5. The implantable system according to claim 4, wherein the prolonged period exceeds a week.

6. The implantable system according to claim 4, wherein the prolonged period exceeds a month.

7. The implantable system according to claim 4, wherein the prolonged period is long is relation to a period required for biofilm to be attached, at an absence of the film, to an amount that prevents the imaging element to image the tissue.

8. The implantable system according to claim 1, wherein the porous enclosure is arranged to prevent tissue liquids to enter the porous inflatable enclosure while allowing an organic liquid composition to flow outside the porous inflatable enclosure.

9. The implantable system according to claim 1, wherein the liquid is an organic liquid composition and wherein the liquid prevents an attachment of biofilm on at least the portion of the porous inflatable enclosure.

10. The implantable system according to claim 1, wherein the liquid is an organic liquid composition that is at least partially transparent and has a density, at 37 degrees Celsius that ranges between 0.65 and 0.9 gram per cubic centimeter.

11. The implantable system according to claim 1, wherein the liquid is an organic liquid composition that is at least partially transparent and is inert.

12. The implantable system according to claim 1, wherein the liquid is a mineral oil.

13. The implantable system according to claim 1, wherein the liquid is an organic liquid composition that comprises linear aliphatic molecules with a carbon atom count that ranges between 8 and 25.

14. The implantable system according to claim 1, wherein the liquid is a liquid organic composition that encapsulates gas bubbles.

15. The implantable system according to claim 1, wherein the porous inflatable enclosure is arranged to facilitate the flow of liquid to maintain a film that is developed over the entire porous inflatable enclosure.

16. The implantable system according to claim 1, wherein the porous inflatable enclosure is shaped and sized to prevent a sealing of a urinary bladder of the person once the implantable system is implanted in the urinary bladder.

17. The implantable system according to claim 1, wherein the porous inflatable enclosure is made of a hydrophobic membrane.

18. The implantable system according to claim 1, wherein hole of the porous inflatable enclosure are evenly spaced over the porous inflatable enclosure.

19. The implantable system according to claim 1, wherein the liquid is a medical drug.

20. The implantable system according to claim 1 further comprising a magnetizable element.

21. The implantable system according to claim 1, wherein the implantable system has a relative density that is smaller than a relative density of urine.

22. The implantable system according to claim 1, wherein at least a portion of an external surface of the porous inflatable enclosure has a texture that is shaped to assist in the generation of the film.

23. The implantable system according to claim 1, further comprising a measurement element for measuring at least one parameter of a cavity of the person in which the implantable system is inserted to.

24. The implantable system according to claim 1, further comprising a sensor operable for intra-vesicle pressure measurement.

25. The implantable system according to claim 1, comprising a one way valve through which the liquid is inserted to the porous inflatable enclosure.

26. The implantable system according to claim 1, comprising a transparent dome that is coupled to the porous inflatable enclosure and is positioned in front of the field of view of the imaging implantable system.

27. The implantable system according to claim 1, comprising a transmitter for transmitting information obtained by the imaging implantable system to a receiver located outside the person.

28. The implantable system according to claim 1, wherein the imaging element is a video camera, an imager, a CCD camera or a CMOS camera.

29. An implantable system for insertion into a cavity of a person, comprising:
an imaging element; and
a porous inflatable enclosure arranged to enclose the imaging element and to enclose liquid that flows through the porous inflatable enclosure, wherein the flow of liquid reduces an attachment of biofilm on the implantable system, when the implantable system is located in the cavity of the person.

30-56. (canceled)

57. A method for imaging a cavity of a person, comprising:
imaging at least a portion of the cavity of the person by an imaging element that belongs to an implantable system that is inserted in the cavity of the person, wherein the imaging comprises acquiring at least one image through an optical element; and
flowing fluid through a porous inflatable enclose arranged to enclose the imaging element and to enclose liquid that flows through the porous inflatable enclosure and contacts the optical element, wherein the liquid, when in contact with the optical element, reduces an aggregation of non-transparent material on the optical element while the implantable system is located in the cavity of the person.

58-114. (canceled)
